(19) United States
(12) Patent Application Publication
(10) Pub. No.: US 2026/0315063 A1
(43) Pub. Date: Oct. 15, 2026

(54) TISSUE CLOSURE DEVICE AND METHOD OF USING SAME

(71) Applicant: Cilag GmbH International, Zug (CH)

(72) Inventors: Mengli Yang, Shanghai (CN); Sheng Ding, Shanghai (CN); Qinglong Liu, Shanghai (CN); Jie Zhang, Shanghai (CN)

(21) Appl. No.: 18/998,634

(22) PCT Filed: Jul. 27, 2023
(86) PCT No.: PCT/EP2023/070812

§ 371 Date: Jan. 27, 2025

(30) Foreign Application Priority Data
Jul. 28, 2022 (CN) 202210900379.6

(51) Int. Cl. A61B 17/04

(52) U.S. Cl. CPC A61B 17/0469; A61B 17/0467; A61B 17/0482; A61B 17/0485; A61B 17/0491; A61B 2017/0472

(57) ABSTRACT

A tissue closure device includes a main body housing and an access needle. A suture slot and a needle exit hole are provided on a sidewall of the main body housing. The access needle can be preloaded in a chamber, and is provided with a suture carrying portion. When the access needle is in an initial position, the access needle is accommodated in the chamber, and the suture carrying portion and the suture slot are at least partially communicated such that a suture can enter the suture carrying portion via the suture slot. In a suture loading process, the tip of the access needle is concealed in the main body housing, so that an operator can be protected from a puncture wound, and the tip of the access needle is less likely to be contaminated.

### TECHNICAL FIELD

The present invention relates to the field of surgical instruments, and in particular to a tissue closure device and a method of using the same.

### BACKGROUND OF THE INVENTION

For the existing tissue closure devices in surgical instruments, generally, the main body of the closure device is inserted into the human body or animal body tissue first, and then a suture is carried by an access needle into the tissue. After entering the tissue, the suture is fixed on a distal structure of the tissue closure device, and suturing is then completed. Because the access needle is operated outside the tissue closure device, the access needle is likely to interfere with the tissue closure device when carrying the suture into the tissue, which may lead to problems such as inaccurate final arrival position of the access needle, separation of the suture from the access needle, and failure of the suture to be accurately delivered to a predetermined position. Moreover, the feature position for loading a suture in a conventional tissue closure device is often narrow in structure, and the operation of loading the suture can be difficult. In addition, when the suture is loaded, the tip of the access needle is usually exposed, which may pose a puncture risk to the operator and may also have certain hygiene hazards.

Therefore, there is a need to provide a tissue closure device and a method of using the same in order to at least partially address the aforementioned problems.

### BRIEF SUMMARY

The present invention provides a tissue closure device and a method of using the same. An access needle can be preloaded in the tissue closure device, and the tissue closure device and the access needle form an integrated unit which can be delivered to an operator conveniently. In the present invention, the unique arrangement of an access needle and a suture slot of a main body housing enables an operator to conveniently load a suture into the access needle and release the suture from the main body housing, and also facilitates exit of the access needle carrying the suture from the tissue closure device. The entire operating process is fast and the control over the access needle is accurate, thus achieving a better operation effect. In a suture loading process, a tip of the access needle is concealed in the main body housing, so that an operator can be protected from a puncture wound, and the tip of the access needle is less likely to be contaminated.

According to one aspect of the present invention, provided is a tissue closure device, comprising:
a main body housing having a longitudinal axis, the main body housing internally defining a chamber extending along the longitudinal axis, and a suture slot and a needle exit hole being provided on a sidewall of the main body housing; and
an access needle, which is capable of being preloaded in the chamber, is provided with a suture carrying portion close to a tip thereof, is capable of being held in an initial position, and is capable of being actuated distally from the initial position to an actuated position,
wherein when the access needle is in an initial position, the access needle is accommodated in the chamber, and the suture carrying portion and the suture slot are at least partially communicated to enable a suture to enter the suture carrying portion via the suture slot; and when the access needle is in the actuated position, the access needle penetrates through the needle exit hole, and the tip of the access needle is located outside the chamber.

In one embodiment, the suture slot comprises a suture loading slot and a suture releasing slot, the suture loading slot being configured to allow the suture to be loaded from the outside of the tissue closure device into the suture carrying portion of the access needle via the suture loading slot, and the suture releasing slot being communicated between the suture loading slot and the needle exit hole and configured to allow the suture to disengage from the tissue closure device via the suture releasing slot, wherein the suture loading slot and the suture carrying portion are at least partially aligned when the access needle is in the initial position.

In one embodiment, the suture slot is an integrated slot serving as both a suture loading slot and a suture releasing slot, the suture slot being configured to allow the suture to be loaded from the outside of the tissue closure device into the suture carrying portion of the access needle via the suture slot and to allow the suture to disengage from the tissue closure device via the suture slot.

In one embodiment, the suture loading slot has a first slot portion and a second slot portion, the first slot portion extending at least partially towards the axis from the outside to the second slot portion, and the second slot portion extending at least partially axially from the first slot portion along a direction away from the needle exit hole, wherein the second slot portion and the suture carrying portion are at least partially aligned.

In one embodiment, the first slot portion constitutes an elongated opening along a direction at least partially perpendicular to the longitudinal axis, two tips of the elongated opening respectively extending along a direction away from the needle exit hole and towards the axis to jointly define the second slot portion.

In one embodiment, the extending directions of the suture releasing slot and the second slot portion are the same.

In one embodiment, a cutting surface that constitutes a guide structure is provided at an inlet of the slot on the sidewall of the main body housing.

In one embodiment, the needle exit hole is an opening fitting the shape of the access needle.

In one embodiment, two perforated structures that are symmetrical about the longitudinal axis are provided on the sidewall of the main body housing, each of the perforated structures comprising at least one said suture slot and at least one said needle exit hole.

In one embodiment, the main body housing comprises a non-rigid suture retaining portion configured to be capable of contacting with the suture and holding the suture in place by friction.

In one embodiment, the main body housing comprises a rigid sidewall having a window formed thereon, the suture retaining portion being mounted at the window and constituting a part of the sidewall of the main body housing, wherein at least a part of the suture slot is at least formed on the suture retaining portion.

In one embodiment, the suture slot comprises a suture loading slot, and a suture releasing slot communicated between the suture loading slot and the needle exit hole, wherein:
at least a part of the suture loading slot is formed on the suture retaining portion; and/or
at least a part of the suture releasing slot is formed on the suture retaining portion.

In one embodiment, a protrusion portion protruding along the sidewall of the main body housing is provided on the suture retaining portion, a corresponding recess portion is provided at the window of the rigid sidewall, and the protrusion portion and the recess portion cooperate to secure the suture retaining portion and the rigid sidewall.

In one embodiment, a protrusion portion protruding along the sidewall of the main body housing is provided at the window of the rigid sidewall, a corresponding recess portion is provided at the edge of the suture retaining portion, and the protrusion portion and the recess portion cooperate to secure the suture retaining portion and the rigid sidewall.

In one embodiment, the portion serving as the suture loading slot of the suture slot is at least partially formed on the suture retaining portion, and has a width less than or equal to the width of the suture.

In one embodiment, the main body housing comprises a rigid sidewall, the suture retaining portion is mounted on the rigid sidewall and is independent of the suture slot, and the suture retaining portion is configured to allow the suture to enter between the suture retaining portion and the rigid sidewall so as to clamp the suture between the suture retaining portion and the rigid sidewall.

In one embodiment, the suture retaining portion is mounted on the side of the suture loading slot away from the needle exit hole.

In one embodiment, two suture slot structures are provided on the sidewall of the main body housing, each of the suture slot structures comprises at least one said suture slot, and the suture retaining portion is located between the two suture slot structures.

In one embodiment, a recessed portion is provided on the rigid sidewall, the suture retaining portion is inserted into the recessed portion with the shape thereof fit to the same, and the outer surface of the suture retaining portion is flush with the outer surface of the rigid sidewall.

In one embodiment, the suture retaining portion comprises a central portion and end portions located at both ends of the central portion, the inner surface of the central portion protruding further inwardly toward the longitudinal axis relative to the inner surfaces of the end portions.

In one embodiment, the suture slot is formed on the rigid sidewall of the main body housing, and the portion serving as the suture loading slot of the suture slot has a width greater than or equal to the width of the suture carrying portion of the access needle.

In one embodiment, the boundary of the suture loading slot has a flared profile relative to the center of the suture loading slot.

In one embodiment, the flared profile is a curved profile.

In one embodiment, a recessed and externally open suture accommodating portion that extends parallel to the longitudinal axis is provided on the sidewall of the main body housing, the suture accommodating portion being connected between the suture slot and a suture winding portion for winding the suture.

In one embodiment, the suture winding portion is mounted in a recess structure on the main body housing, the suture winding portion comprises a mounting base portion directly joined to the main body housing and a suture winding portion body located on the outer side of the mounting base portion, the mounting base portion and the suture winding portion body are connected by a thin neck portion, and the suture is wound around the thin neck portion.

In one embodiment, a recessed groove extending circumferentially is provided on the sidewall of the main body housing to form the suture winding portion.

In one embodiment, the tissue closure device comprises a stabilizer mounted at one end of the main body housing and actuatable between a folded position and an unfolded position, and when the access needle is in the actuated position, the access needle penetrates through the stabilizer in the unfolded position.

According to another aspect of the present invention, provided is a method of using the tissue closure device according to any one of the above solutions, comprising:
a suture loading step comprising: loading a suture into a suture carrying portion of an access needle via a suture loading slot of a main body housing; and
a needle exiting step comprising: causing the access needle loaded with the suture to extend outwardly from a needle exit hole of the main body housing to an actuated position.

### BRIEF DESCRIPTION OF DRAWINGS

To better understand the above and other objectives, features, advantages and functions of the present invention, reference may be made to preferred embodiments shown in the accompanying drawings. The same or similar reference numerals refer to the same or similar components in the accompanying drawings. It should be understood by those skilled in the art that the drawings are intended to schematically illustrate the preferred embodiments of the invention and have no limitation on the scope of the invention, and various members are not drawn to scale.

FIG. 1 is a schematic perspective view of a tissue closure device according to a preferred embodiment of the present invention, wherein an access needle is in an initial position and a stabilizer is in a folded position;

FIG. 2 is a partially enlarged schematic diagram of the structure at a suture slot of a main body housing in FIG. 1;

FIG. 3 is a schematic section view of FIG. 2, wherein the exterior structure of the main body housing is partially omitted to show the access needle;

FIG. 4 is a schematic diagram of the structure at the suture slot in FIG. 2 taken from another angle of view;

FIG. 5 is a schematic diagram of the structure at the suture slot in FIG. 2 taken from still another angle of view;

FIG. 6 is a schematic perspective view in which the main body housing in FIG. 1 is omitted;

FIG. 7 is a schematic diagram of the tissue closure device in FIG. 1 in another state, wherein the access needle is in an actuated position and the stabilizer is in an unfolded position;

FIG. 8 is a schematic perspective view in which the main body housing in FIG. 7 is omitted;

FIG. 9 is a schematic diagram showing cooperation of a needle control component and a stabilizer control component in FIG. 8;

FIG. 10 is a schematic section view of FIG. 9, which shows the interior structure of the needle control component;

FIG. 11 is a schematic exploded perspective view of the needle control component, the access needle, and a fixing pin in FIG. 10;

FIG. 12 is a schematic diagram of another embodiment of the present invention, which only shows the structure at a suture slot of a main body housing;

FIG. 13 is a schematic diagram of FIG. 12 taken from another angle of view;

FIG. 14 is a schematic diagram of still another embodiment of the present invention, which only shows the structure at and near a suture slot of a main body housing;

FIG. 15 is a schematic diagram of yet another embodiment of the present invention, which only shows the structure at a suture slot and a suture winding portion of a main body housing;

FIG. 16 is a schematic diagram of an alternative embodiment of FIG. 15, which also only shows the structure at a suture slot and a suture winding portion of a main body housing;

FIGS. 17 and 18 are schematic diagrams of a suture slot portion according to yet another embodiment of the present invention, wherein FIGS. 17 and 18 are taken from different angles of view, and FIG. 18 is a partial view; and

FIG. 19 is a section view of FIG. 18, wherein the structure of a main body housing is partially omitted to show an access needle.

### DESCRIPTION OF REFERENCE NUMERALS

100 Tissue closure device
10, 610, 710, 810, 910, 1010 Main body housing
11 Housing joining line
12, 1012 Suture slot
121, 6121, 7121, 8121, 9121 Suture loading slot
1211, 10121 First slot portion
1212, 10122 Second slot portion
1212a Flared profile
122, 6122, 7122, 8122, 9122 Suture releasing slot
14 Left half housing
15 Right half housing
16, 616, 716, 816, 916, 1016 Needle exit hole
17 Needle channel
618 Cutting surface
101 First window
102 Second window
20 Needle control component
21 Needle operation portion
22 Needle actuation portion
221 Radially recessed section
222 Access needle mounting hole
223 Fixing pin
2231 Fixing pin hole
224 Position-limiting slot
30 Stabilizer control component
31 Stabilizer operation portion
32 Positioning protrusion
33 Stabilizer actuation portion
40 Stabilizer
41 Shield
42 Soft rubber block
50, 1050 Access needle
51, 1051 Suture carrying portion
52 Tip of access needle
55 Notch
660, 760, 1060 Suture retaining portion
661 Protrusion portion
711 Recessed portion
761 Central portion
762 End portion
811, 911 Suture winding portion
8111 Mounting base portion
8112 Suture winding portion body
8113 Thin neck portion
813, 913 Suture accommodating portion
8131, 9131 Tip of suture accommodating portion
814 Recess structure

### DETAILED DESCRIPTION

A tissue closure device and a method of using the same according to the present invention are described in detail below with reference to the accompanying drawings. The embodiments described below are merely preferred embodiments according to the present invention, and on the basis of the preferred embodiments, those skilled in the art can conceive of other modes capable of implementing the present invention, which also fall within the scope of the present invention.

The present invention provides a tissue closure device for surgical procedures. First of all, it should be noted that the directional terms and position terms mentioned in the present invention should be construed as relative directions and relative positions. For example, “axis direction” and “axial direction” and the like mentioned in the present invention can be construed as the direction along the axis of the main body housing; “radial direction”, “circumferential direction” and the like are radial and circumferential directions with respect to the axis; “distal end” and “distal” refer to the direction along the axis and away from the operator (e.g. a surgeon); and “proximal end” and “proximal” refer to the direction along the axis and close to the operator (e.g. a surgeon).

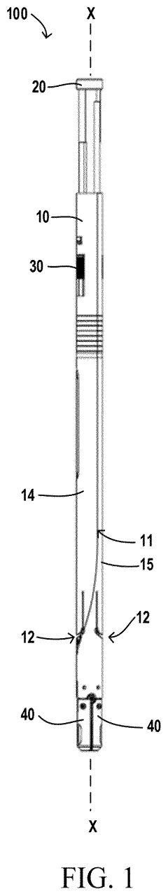

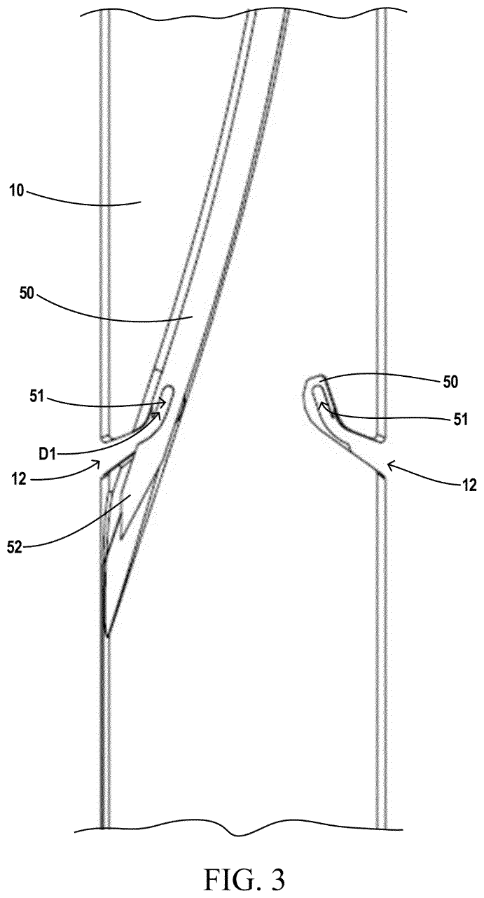

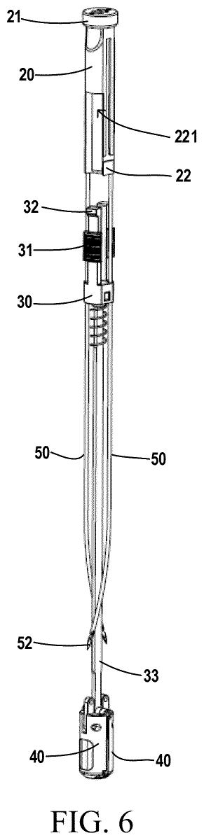

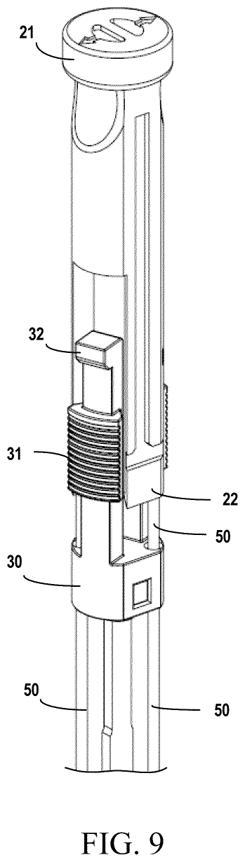

FIGS. 1 to 11 show one preferred embodiment of the present invention. First referring to FIG. 1, a tissue closure device 100 includes a main body housing 10 having a longitudinal axis, and the main body housing 10 internally defines a chamber extending along the longitudinal axis. The chamber is preloaded with an access needle 50 therein (for example, referring to the view in FIG. 6 with the main body housing 10 removed) and has a needle channel (referring to FIG. 3) for the access needle 50 to move. The access needle 50 is used to carry a suture into the tissue of a patient. The tissue closure device 100 further includes a needle control component 20 mounted at a proximal portion thereof, and the needle control component 20 is fixedly connected to a proximal end of the access needle 50. The needle control component 20 can move axially relative to the main body housing 10, thereby controlling the access needle 50 to move from an initial position to an actuated position.

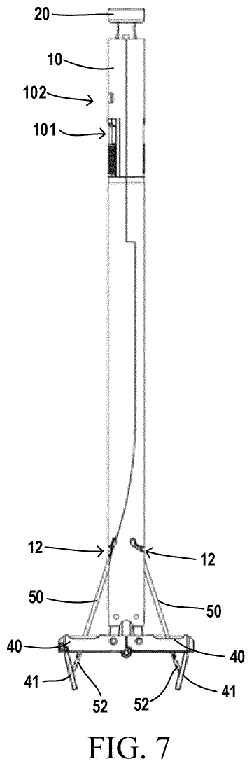

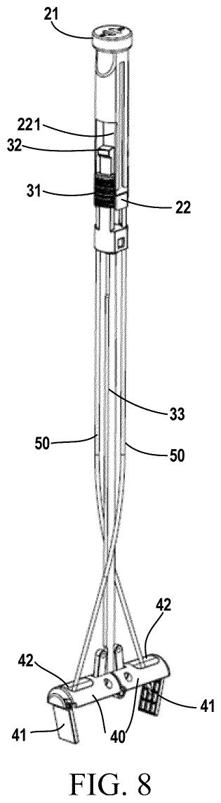

Reference may be made to FIGS. 1 to 6 for the state of the access needle 50 in the initial position, and reference may be made to FIGS. 7 and 8 for the state of the access needle 50 in the actuated position. When the access needle 50 is in the initial position relative to the main body housing 10, the access needle 50 is entirely located in the chamber of the main body housing 10, and a distal end 52 of the access needle 50 is in the most proximal position relative to the main body housing 10. When the access needle 50 is in the actuated position relative to the main body housing 10, the distal end 52 of the access needle 50 is located outside the main body housing 10 and the distal end 52 of the access needle 50 is in the most distal position relative to the main body housing 10.

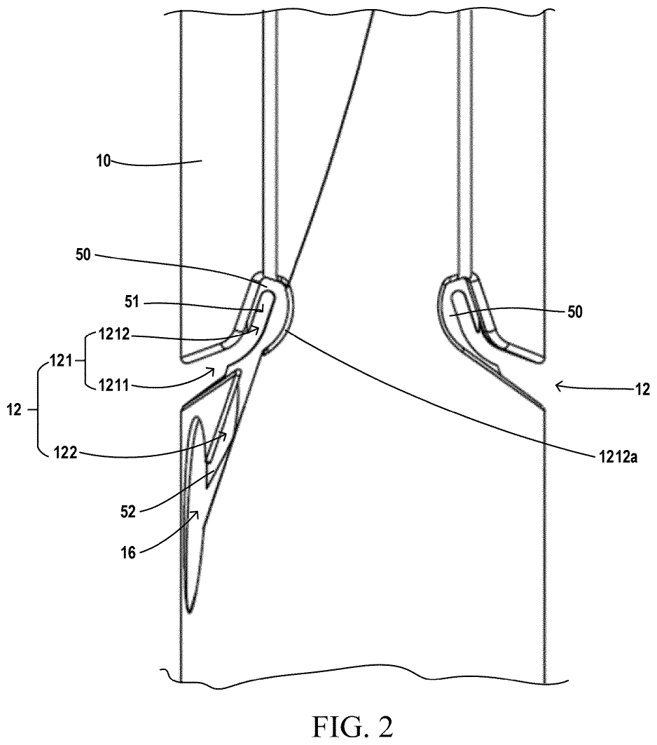

Also referring to FIGS. 2 to 5, a suture slot 12 that communicates the chamber of the main body housing 10 with the outside is provided on a sidewall of the main body housing 10, and the suture slot 12 at least partially extends along the radial direction of the main body housing 10. The access needle 50 is formed with a suture carrying portion 51 close to the distal end 52 thereof, and the suture carrying portion 51 is also in the form of a slot structure extending at least partially proximally along the extending direction of the access needle 50. The needle control component 20 can hold the access needle 50 in the initial position in a predetermined orientation. When the access needle 50 is held in the initial position, the distal end 52 of the access needle 50 is located in the chamber. The “predetermined orientation” means that the orientation of the access needle in the initial position enables the suture slot 12 of the main body housing 10 and the suture carrying portion 51 of the access needle 50 to be at least partially communicated, such that the operator can load a suture into the suture carrying portion 51 of the access needle 50 via the suture slot 12 of the main body housing 10 (it can be appreciated that if the access needle 50 is rotated with respect to the predetermined orientation, the suture carrying portion 51 may not communicate with the slot 12, resulting in failure to successfully load the suture). For example, the inner wall of the suture slot 12 is defined to have a flat surface, such that the access needle 50 is fit to the flat surface and prevented from twisting in the suture slot 12, thus ensuring partial communication between the suture carrying portion 51 and the suture slot 12.

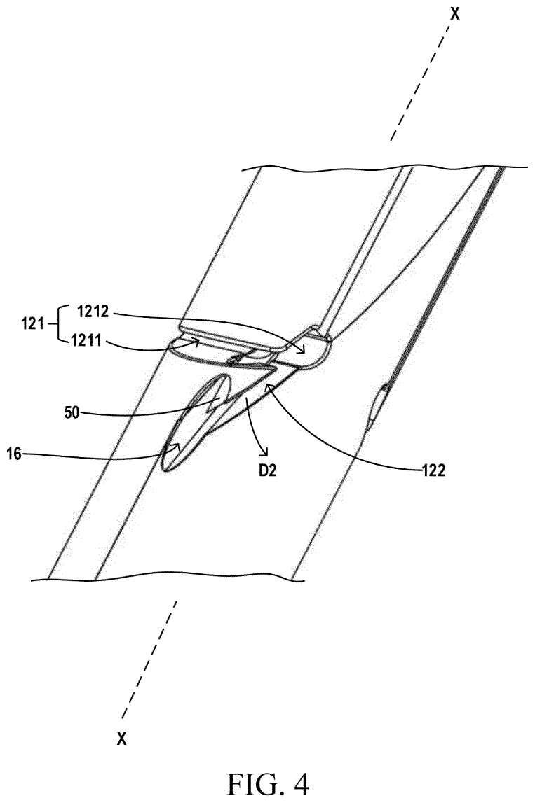

The suture slot 12 may include, for example, a suture loading slot 121 and a suture releasing slot 122. The suture releasing slot 122 may, for example, communicate at a distal end of the suture loading slot 121 and a distal end of the suture releasing slot 122 may further communicate with a needle exit hole 16. The needle control component 20 can control the access needle 50 to extend to an actuated position at a predetermined depth. In the process of the access needle 50 moving from the initial position to the actuated position, the distal end 52 of the access needle 50 will extend through the needle exit hole 16 and exit the main body housing 10. The portion serving as the suture loading slot 121 of the suture slot 12 is used to load a suture into the suture carrying portion 51 from the outside of the tissue closure device 100 when the access needle 50 is held in the initial position in the predetermined orientation, wherein the movement direction of the suture in this process is shown by, for example, an arrow D1 in FIG. 3. The portion serving as the suture releasing slot 122 of the suture slot 12 is used to allow the suture to exit the tissue closure device via the suture releasing slot 122 after the access needle has been moved to the actuated position via the needle exit hole 16, wherein the movement direction of the suture in this process is shown by, for example, an arrow D2 in FIG. 4.

Further, the suture loading slot 121 has a first slot portion 1211 and a second slot portion 1212. The first slot portion 1211 extends at least partially towards the axis from the outside to the second slot portion 1212, and the second slot portion 1212 extends at least partially axially proximally from the first slot portion 1211, wherein the second slot portion 1212 and the suture carrying portion 51 are at least partially aligned. For example, as shown in FIGS. 2 and 3, when the access needle 50 is in the initial position, the extending directions of the second slot portion 1212 and the suture carrying portion 51 of the access needle 50 are substantially the same, as can be seen from a side view of the tissue closure device 100. More specifically, in this embodiment, the first slot portion 1211 extends both radially inward and axially proximally, and the second slot portion 1212 also extends radially inward and axially proximally. An included angle between the extending direction of the second slot portion 1212 and the longitudinal axis is less than an included angle between the first slot portion 1211 and the longitudinal axis.

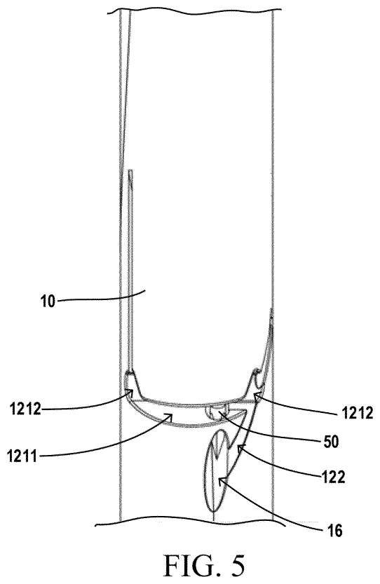

Also referring to FIGS. 4 and 5, the first slot portion 1211 constitutes an elongated opening formed on the sidewall in a direction perpendicular to the longitudinal axis, and two tips of the elongated opening respectively extend proximally and towards the axis to jointly define the second slot portion 1212. The extending directions of the suture releasing slot 122 and the second slot portion 1212 are substantially the same, and the suture releasing slot 122 smoothly transitions from the second slot portion 1212 to the needle exit hole 16. It can be clearly seen from FIGS. 4 and 5 that, the needle exit hole 16 is an opening fitting the shape of the access needle 50, and the opening may have, for example, a round or oval contour.

Because the suture slot 12 is formed on the main body housing 10 made of a rigid material in this embodiment, the suture slot 12 needs to have a certain width to allow the suture to pass through. For example, the width of the suture loading slot 121 is greater than or equal to the width of the suture carrying portion 51 of the access needle 50. Preferably, the suture loading slot 121 may have a clearance design at the boundary, so that after the suture enters the suture loading slot 121, the suture carrying portion 51 can also be pressed to drive the distal end of the access needle 50 to move in the suture slot 12 in a very small range, thus more accurately completing suture loading of the access needle 50. The clearance design may include, for example, a flared profile 1212a relative to the center of the suture loading slot, that is formed on the boundary of the second slot portion 1212 of the suture loading slot 121, and the flared profile 1212a may be, for example, a curved segment. The clearance design may also have other forms.

Referring to FIG. 1, and 6 to 8 as a whole, in this embodiment, two access needles 50 are provided, and the proximal ends of the two access needles 50 are fixed on the same needle control component 20, so that the needle control component 20 controls the two access needles 50 simultaneously. The access needle 50 is easily bendable and flexible, and is made of, for example, a plastic material. The chamber in the main body housing 20 includes a needle channel which is configured to enable the access needle 50 to move therein in a predetermined pose. In other words, the form of the needle channel determines the form of the access needle 50. It can be seen from FIGS. 6 and 8 that, in the initial position, each access needle 50 may include, for example, a straight segment and a curved segment located at the distal end of the straight segment, and the curved segment extends distally partially about the longitudinal axis. Then, it can be understood that, in fact, it is the needle channel that has such straight and curved segments. In the actuated position, the access needle 50 may include two straight segments and a curved segment between the two straight segments. The curved segment extends partially about the longitudinal axis, the straight segment located proximally of the curved segment extends axially, and the straight segment located distally of the curved segment extends both axially and radially. Likewise, it can be understood that, in fact, it is the needle channel that has such straight and curved segments (certainly, except for the portions outside the main body housing).

Also referring to FIG. 8, under the operation of the needle control component 20 and the position-limiting effect of the needle channel, when the distal ends 52 of the access needles 50 reach the actuated position, a connecting line between the distal ends 52 of the two access needles 50 is in a radial direction with respect to the axis, and the radial direction is, for example, also the extending direction of a stabilizer 40 (which will be described in detail later).

The two needle channels are symmetrical about the axis at any axial position. As shown in FIGS. 6 and 8, such arrangement enables the portions of the two access needles 50 at any axial position to be symmetrical about the longitudinal axis. Correspondingly, two perforated structures are correspondingly provided on the sidewall of the main body housing 10, and each of the perforated structures includes a suture slot 12 and a needle exit hole 16. The two perforated structures are substantially symmetrical about the axis. For example, the corresponding parts of the two perforated structures are symmetrical about the axis. Further, for example, the respective suture loading slots 121 of the two perforated structures are symmetrical about the axis, the respective suture releasing slots 122 are symmetrical about the axis, and the respective needle exit holes 16 are symmetrical about the axis.

Referring to FIGS. 1, 7 and 8, the tissue closure device 100 further includes a stabilizer 40, and a stabilizer control component 30 mounted on the main body housing 10 close to a proximal end thereof. The stabilizer 40 includes, for example, at least two wing portions that are located on a distal end of the main body housing 10. The stabilizer control component 30 can move axially with respect to the main body housing 10 in response to operation of an operator, thereby driving the at least two wing portions to be actuated between a folded position and an unfolded position. For example, when the stabilizer control component 30 moves distally relative to the main body housing 10, the stabilizer 40 is actuated from the folded position to the unfolded position. In the state shown in FIG. 1, the stabilizer 40 is in the folded position. The stabilizer 40 in the folded position extends substantially axially distally from the main body housing 10, and the radial dimension of the stabilizer 40 in the folded position is approximately equal to the outer diameter of the main body housing 10. In the states shown in FIGS. 7 and 8, the stabilizer 40 is in the unfolded position, and the extending direction of the stabilizer 40 in the unfolded position is substantially perpendicular to the longitudinal axis. The stabilizer 40 may be used as, for example, a depth limiting mechanism. When the distal end of the tissue closure device 100 extends to approximately a predetermined depth within the patient, the stabilizer control component 30 may be operated to unfold the stabilizer 40. The proximal surface of the unfolded stabilizer 40 is fit to the inner wall of the tissue of the patient, thereby controlling the extension of the tissue closure device 100 to the current depth within the patent.

A soft rubber block 42 may be disposed on the stabilizer 40. In the process of the access needle 50 moving from the initial position to the actuated position, the distal end 52 of the access needle 50 will pass through the soft rubber block 42 and reach the distal position of the stabilizer 40. Preferably, in order to prevent the distal end 52 of the access needle 50 from piercing the tissue of the patient, a shield 41 extending distally from the stabilizer 40 may be provided at a radial outer side of the stabilizer 40. When the access needle 50 is in the actuated position, the shield 41 provides protection at a radial outer side of the distal end 52 of the access needle 50. Additionally or alternatively to the above preferred arrangements, this embodiment may also have preferred arrangements in some aspects. The preferred arrangements will be discussed in detail below with reference to FIG. 1, and 7 to 11.

For example, this embodiment may have some preferred arrangements in terms of the form of the needle control component 20, the joining manner between the needle control component 20 and the main body housing 10, and the connection manner between the needle control component 20 and the access needle 50. The needle control component 20 has a needle operation portion 21 located outside the main body housing 10 for a user to operate, and a needle actuation portion 22 located in the chamber and connected to the access needle 50. The chamber of the main body housing 10 may have a proximal opening from which the needle operation portion 21 extends to form a button structure for the operator to press. Further, a position-limiting structure is provided at the connection position between the needle control component 20 and the main body housing 10, and the position-limiting structure only allows the needle control component 20 to move axially relative to the main body housing 10 and prevents the needle control component 20 from rotating or translating in other directions relative to the main body housing 10.

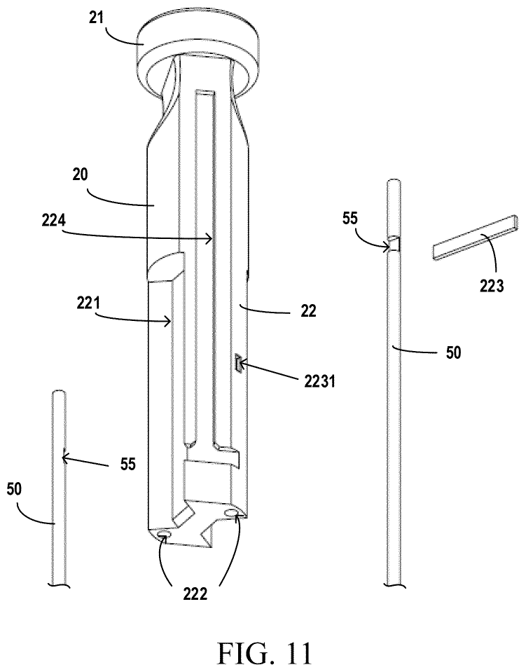

The position-limiting structure includes, for example, an axially extending position-limiting slot 224 (as shown in FIG. 11) located on an outer surface of the actuation portion, and a protrusion located on an inner surface of the main body housing 10 and having a width matching that of the position-limiting slot 224. In this embodiment, the main body housing 10 includes a left half housing 14 and a right half housing 15, and the left half housing 14 and the right half housing 15 are butted together along a direction transverse to the longitudinal axis, with a housing joining line 11 formed at the connection position therebetween. The left half housing 14 and the right half housing 15 are respectively provided with protruding halves at portions thereof adjacent to each other. The protruding halves of the left half housing 14 and the right half housing 15 are butted together to jointly define the protrusion.

It can be understood that, in this embodiment, the protrusion can slide along the position-limiting slot 224 so that the needle control component 20 moves from a first position (for example, a proximal position) to a second position (for example, a distal position) relative to the main body housing 10. When the needle control component 20 is located at the first position, the needle control component 20 holds the access needle 50 in the initial position in a predetermined orientation. When the needle control component 20 moves from the first position to the second position, the access needle 50 is driven to move from the initial position to the actuated position at a predetermined depth.

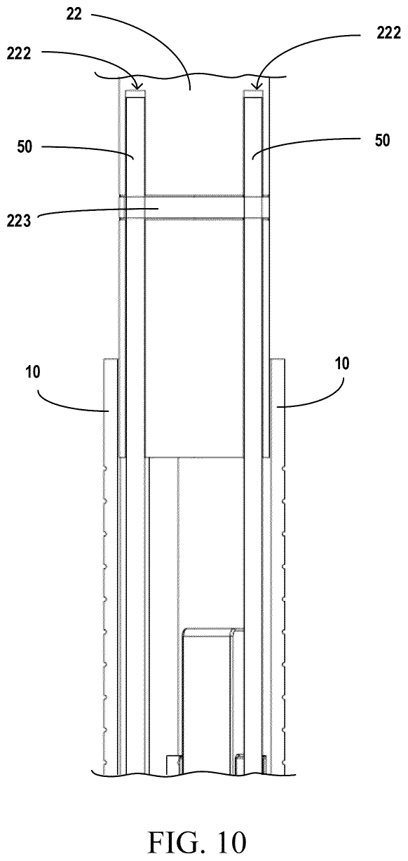

Referring to FIGS. 10 and 11, the needle actuation portion 22 has two holes 222 that are open distally and extend axially, and the inner diameter of each hole 222 matches the outer diameter of one access needle 50. And a fixing pin 223 transverse to the longitudinal axis is provided in the needle control component 20 and the fixing pin 223 is connected between the two holes 222. Each access needle 50 may be provided with a notch 55 for receiving the fixing pin 223, and the fixing pin 223 is engaged and fixed at the notch 55. That is to say, one fixing pin 223 is joined to two access needles 50 simultaneously, so that the two access needles 50 are fixed to the needle actuation portion 22 simultaneously. The needle actuation portion 22 is provided with a fixing pin hole 2231 for insertion of the fixing pin 223 in an assembly stage.

The wall surface of the notch 55 may include a flat surface, so as to enhance the joining stability between the fixing pin 223 and the access needle 50. The notch 55 may extend in the middle portion of the access needle 50, and may also extend all the way to the proximal tip of the access needle 50. It should be noted that, the notch is only an example of the “mating portion”, and the mating portion may also have other forms in other embodiments that are not shown. In some embodiments, the fixing pin may not be provided, and the mating portion is directly molded together with the needle control component.

This embodiment may also have some preferred arrangements in terms of the connection manner between the stabilizer control component 30 and the main body housing 10, and the connection manner between the stabilizer control component 30 and the needle control component 20. For example, the sidewall of the main body housing 10 is provided with a first window 101 and a second window 102 located proximally of the first window 101 (referring to FIG. 7). The stabilizer control component 30 includes a stabilizer operation portion 31 exposed through the windows, a positioning protrusion 32 located proximally of the stabilizer operation portion 31 and fixed relative to the stabilizer operation portion 31, and a stabilizer actuation portion 33 located distally of the stabilizer operation portion 31. A radial outer portion of the stabilizer operation portion 31 has an axial dimension smaller than that of the first window 101 so as to be able to axially slide in the first window 101. The size of a radial outer portion of the positioning protrusion 32 matches that of the second window 102. When the positioning protrusion 32 is placed in the second window 102, the stabilizer control component 30 is locked relative to the main body housing 10, for example, as shown in FIG. 1. When the positioning protrusion 32 is located in the second window 102, the operator is allowed to press the positioning protrusion 32 radially inwardly, and the positioning protrusion 32 is biased inwardly to disengage from the second window 102 to unlock the stabilizer control component. Subsequently, the positioning protrusion 32 can be actuated distally together with the stabilizer operation portion 31. When the stabilizer control component 30 actuates the stabilizer 40 to the unfolded position, the positioning protrusion 32 is positioned in the first window 101 together with the stabilizer operation portion 31, as shown in FIGS. 7 and 8. Preferably, the axial dimension between the most proximal end of the positioning protrusion 32 and the most distal end of the stabilizer operation portion 31 is just equal to the axial dimension of the first widow 101, such that the stabilizer control component 30 cannot move axially relative to the main body housing 10 when the positioning protrusion 32 is located in the first window 101 together with the stabilizer operation portion 31.

Further, there are two stabilizer operation portions 31. The stabilizer control component 30 further includes a stabilizer actuation portion 33 fixedly connected at the distal ends of the two stabilizer operation portions 31. A radially recessed section 221 (as shown in FIG. 11) is provided on a sidewall of the needle control component 20, and at least a radial inner portion of the stabilizer operation portion 31 is correspondingly mounted at the radially recessed section 221. The needle control component 20 is partially located between the two stabilizer operation portions 31 and is completely located proximally of the stabilizer actuation portion 33.

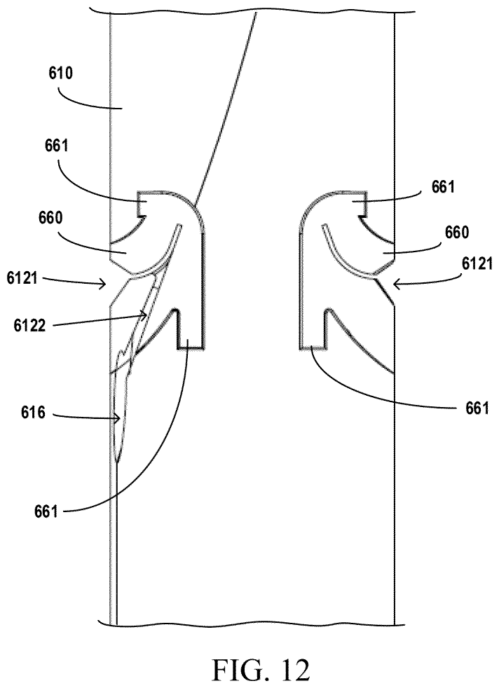

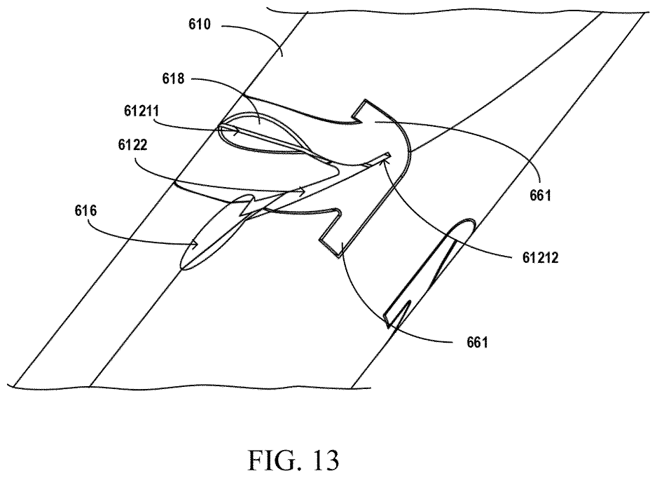

FIGS. 12 and 13 show the structure at the suture slot of the tissue closure device according to another preferred embodiment of the present invention. A main body housing 610 includes a rigid sidewall and a suture retaining portion 660 that can provide greater friction. The suture retaining portion 660 is made of, for example, a flexible, plastic, or elastic material. In an embodiment, the suture retaining portion 660 may be made of a soft rubber. The suture retaining portion 660 can hold the suture carried by the access needle in place by means of friction when the access needle is in the initial position. For example, a window is formed on the rigid sidewall, and the suture retaining portion 660 is mounted at the window and constitutes a part of the sidewall of the main body housing 610, wherein at least a part of the suture slot is formed on the suture retaining portion 660.

As shown in FIGS. 12 and 13, a suture loading slot 6121 and a suture releasing slot 6122 are both formed on the suture retaining portion 660, and a needle exit hole 6122 is jointly defined by the suture retaining portion 660 and the rigid sidewall. In this embodiment, the suture loading slot 6121 also includes a first slot portion 61211 and a second slot portion 61212, and cutting surfaces 618 that function as a guide structure are formed at an inlet of the first slot portion 61211. The cutting surfaces 618 are provided proximally and distally of the inlet respectively, to facilitate loading of the suture from the outside via the suture slot into the suture carrying portion of the access needle.

In this embodiment, because the suture retaining portion 660 can provide friction to the suture, a suture slot 612 formed on the suture retaining portion 660 can have a smaller width. For example, the width of the suture slot 612 can be smaller than the width of the suture slot in the embodiment shown in FIGS. 1 to 11. Further, the width of the suture slot 612 can be even smaller than or equal to the width of the suture, such that when the suture enters the suture slot 612, an interference fit is created between the suture and the suture slot 612, and a slight deformation may occur at the suture slot 612. This slight deformation does not affect the loading of the suture, but can help to clamp the suture, avoiding the suture from coming off. It can be understood that, if the suture slot is partially defined by the suture retaining portion (for example, the suture is jointly defined by the suture retaining portion and the rigid sidewall), the suture slot can still have such a smaller width.

Preferably, the suture retaining portion 660 is provided with a protrusion portion 661 protruding along the sidewall of the main body housing 610, a corresponding recess portion is provided at the window of the rigid sidewall, and the protrusion portion 661 and the recess portion cooperate to secure the suture retaining portion 660 and the rigid sidewall. Additionally or alternatively to this solution, a protrusion portion protruding along the sidewall of the main body housing is provided at the window of the rigid sidewall, a corresponding recess portion is provided at the edge of the suture retaining portion, and the protrusion portion and the recess portion cooperate to secure the suture retaining portion and the rigid sidewall.

It should be noted that, in addition to the foregoing structures, this embodiment is identical to or similar to the embodiment shown in FIGS. 1 to 11, and the description of the embodiment in FIGS. 1 to 11 should also be regarded as description of this embodiment.

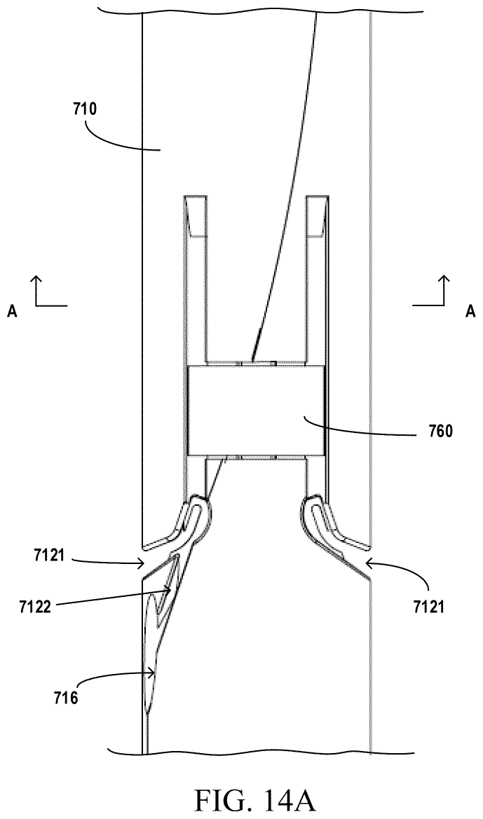

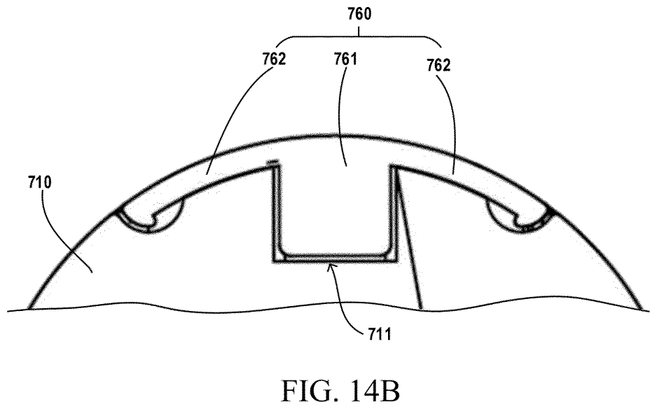

FIGS. 14A and 14B show still another preferred embodiment of the present invention. In this embodiment, a suture retaining portion 760 is different from the suture retaining portion in FIGS. 12 and 13. In the embodiment shown in FIG. 14A, a suture loading slot 7121, a suture releasing slot 7122, and a needle exit hole 716 are provided on the sidewall of the main body housing 710, and the structural forms of the suture loading slot 7121, the suture releasing slot 7122, and the needle exit hole 716 are identical to or similar to those in FIGS. 1 to 11. In FIG. 14, the suture retaining portion 760 is mounted on the rigid sidewall and independent of the suture loading slot 7121, the suture releasing slot 7122, and the needle exit hole 716. The suture retaining portion 760 allows the suture to enter between the suture retaining portion 760 and the rigid sidewall so as to clamp the suture between the suture retaining portion 760 and the rigid sidewall. Preferably, the suture retaining portion 760 may be mounted proximally of the suture loading slot 7121, and located between the two suture loading slots 7121 circumferentially.

Preferably, the outer surface of the suture retaining portion 760 is flush with the outer surface of the rigid sidewall. It can be understood that, in order to achieve this feature, a recessed portion 711 will be provided on the rigid sidewall, and the suture retaining portion 760 is inserted into the recessed portion with the shape thereof fit to the same. More preferably, referring to FIG. 14B, the suture retaining portion includes a central portion 761 and end portions 762 located at both circumferential ends of the central portion 761, and the inner surface of the central portion 761 protrudes further inwardly toward the longitudinal axis relative to the inner surfaces of the end portions 762.

It should be noted that, in addition to the foregoing structures, this embodiment is identical to or similar to the embodiment shown in FIGS. 1 to 11, and the description of the embodiment in FIGS. 1 to 11 should also be regarded as description of this embodiment.

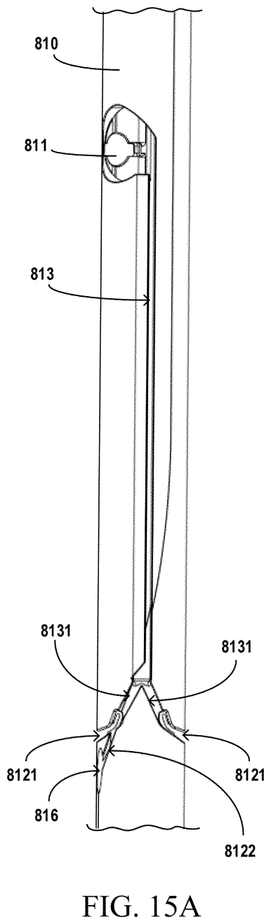

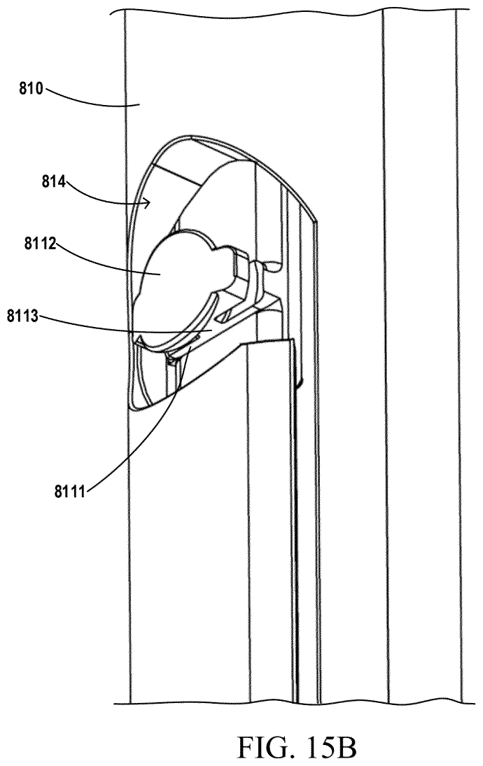

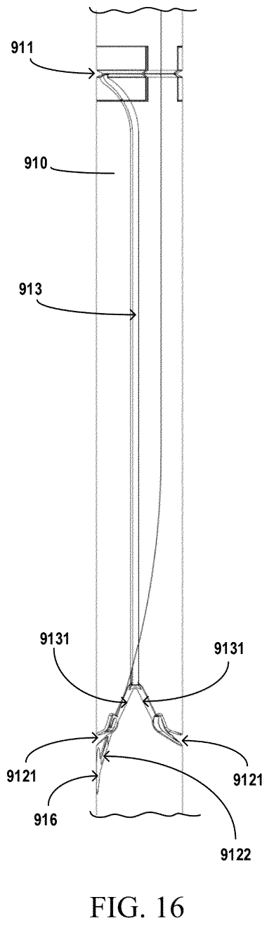

FIGS. 15A to 16 show some other preferred embodiments of the present invention, and mainly illustrate two forms of a suture winding portion provided on a main body sidewall. It should be noted that, the suture winding portion in FIGS. 15A to 16 may be applied to the embodiments in FIGS. 1 to 14.

Referring to FIG. 15A, a suture loading slot 8121, a suture releasing slot 8122, and a needle exit hole 816 that are similar to those in the embodiment of FIGS. 1 to 11 are provided on the sidewall of a main body housing 810. The main body housing is further provided with a recessed and externally open suture accommodating portion 813 that extends parallel to the longitudinal axis, the distal end of the suture accommodating portion 813 communicates with the suture loading slot 8121, and the proximal end of the suture accommodating portion 813 is provided with a suture winding portion 811 for loading the suture. Two branches 8131 are formed at the distal end of the suture accommodating portion 813, so as to communicate with the two suture loading slots 8121, respectively. It can be seen from FIG. 15B that, the suture winding portion 811 is mounted in a recess structure 814 on the main body housing 810, and the suture winding portion 811 is formed into a round button and includes a mounting base portion 8111 directly joined to the main body housing 810 and a suture winding portion body 8112 located on the outer side of the mounting base portion 8111. The mounting base portion 8111 and the suture winding portion body 8112 are connected by a thin neck portion 8113, and the suture is wound around the thin neck portion 8113.

Referring to FIG. 16, a suture loading slot 9121, a suture releasing slot 9122, and a needle exit hole 916 that are similar to those in the embodiment of FIGS. 1 to 11 are provided on a main body housing 910. The main body housing 910 is further provided with a recessed and externally open suture accommodating portion 913 that extends parallel to the longitudinal axis, the distal end of the suture accommodating portion 913 communicates with the suture loading slot 9121, and the proximal end of the suture accommodating portion 913 is provided with a suture winding portion 911 for loading the suture. Two branches 9131 are formed at the distal end of the suture accommodating portion 913, so as to communicate with the two suture loading slots 9121, respectively. The suture winding portion 911 may be a recessed groove extending along the entire circumference.

It should be noted that, various arrangements in this embodiment may be identical to or similar to the embodiment shown in FIGS. 1 to 11, and the description of the embodiment in FIGS. 1 to 11 should also be regarded as description of this embodiment.

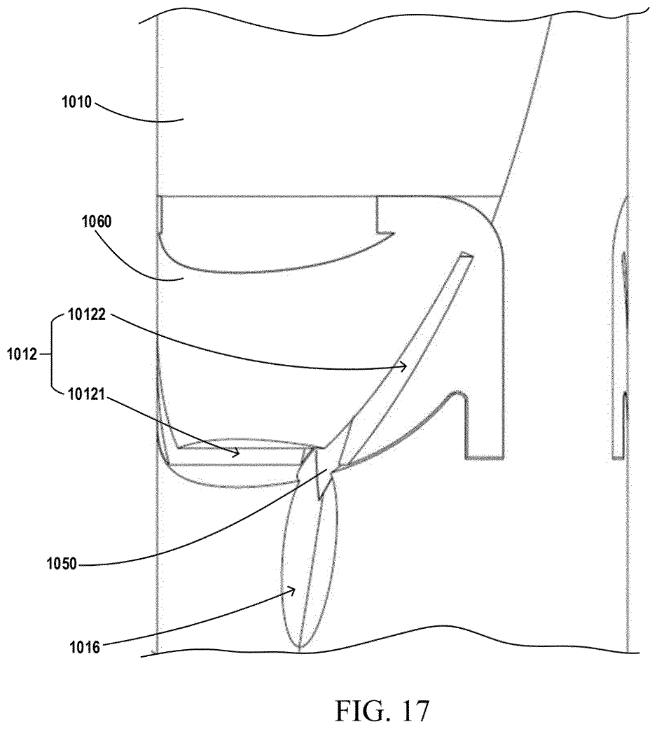

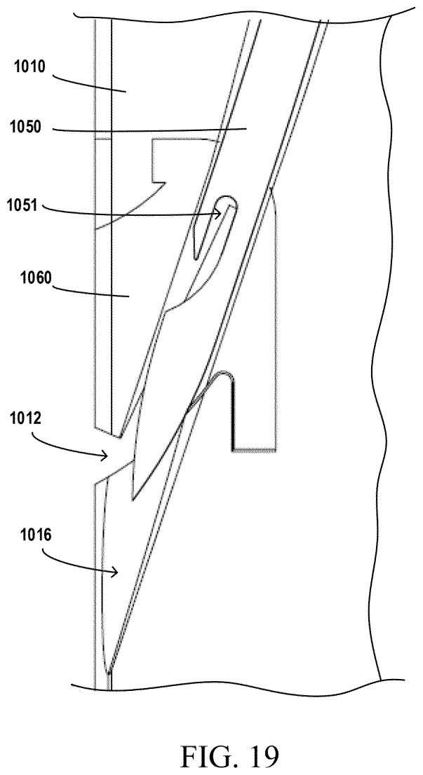

FIGS. 17 to 19 show still another embodiment of the present invention. In this embodiment, the form of the suture slot on the main body sidewall is different from that shown in FIGS. 1 to 16. Referring to FIGS. 17 to 19, a suture slot 1012 is an integrated slot serving as both a suture loading slot and a suture releasing slot. The suture slot 1012 is configured to allow a suture to be loaded from the outside of the tissue closure device via the suture slot 1012 into a suture carrying portion 1051 of an access needle 1050 and to allow the suture to disengage from the tissue closure device via the suture slot 1012. The suture slot 1012 directly communicates with a needle exit hole 1016. Further, the suture slot 1012 is formed on a suture retaining portion 1060 on the main body sidewall, and the needle exit hole 1016 is jointly defined by the suture retaining portion 1060 and the rigid sidewall. The suture slot 1012 also includes a first slot portion 10121 serving as a suture loading inlet, and a second slot portion 10122 extending proximally from the first slot portion 10121 and towards the axis. When a suture is loaded, the suture needs to move proximally along the second slot portion 10122, till the suture reaches the suture carrying portion 1051.

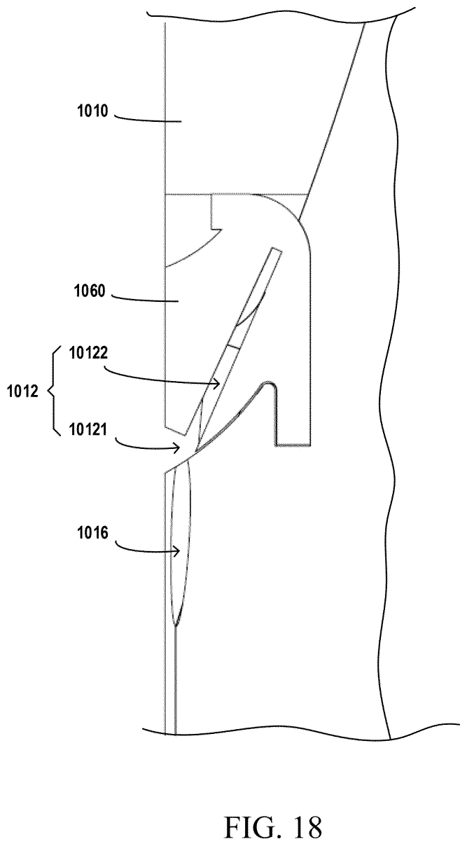

The suture slot includes a first slot portion and a second slot portion. The first slot portion extends at least partially towards the axis from the outside to the second slot portion, and the second slot portion extends at least partially axially proximally from the first slot portion. In the view shown in FIG. 18, the extending length of the second slot portion is several times of the extending length of the first slot portion.

Referring back to FIG. 17, the first slot portion constitutes an elongated opening formed on the sidewall in a direction perpendicular to the longitudinal axis, and two tips of the elongated opening respectively extend proximally and towards the axis to jointly define the second slot portion.

The suture carrying portion of the access needle is concealed in the second slot portion. When a suture is to be loaded, the suture needs to move proximally to the suture carrying portion along the second slot portion. The distal end of the access needle carrying the suture can exit the main body housing via the needle exit hole. In the final step where the suture needs to be released, the suture also exits the main body housing via the integrated suture slot.

It should be noted that, except for the arrangement of the suture slot, various arrangements in this embodiment may be identical to or similar to those in the embodiment shown in FIGS. 1 to 11, and the description of the embodiment in FIGS. 1 to 11 should also be regarded as description of this embodiment.

It should be further noted that, specific arrangements in the foregoing embodiments may be combined to obtain new embodiments, and these new embodiments also fall within the scope of the present invention.

The present invention further provides a method of using the tissue closure device in any one of the foregoing solutions. The method includes a suture loading step and a needle exiting step. The suture loading step includes: loading a suture into a suture carrying portion of an access needle via a suture loading slot of a main body housing. The needle exiting step includes: causing a needle control firmware to drive the access needle loaded with the suture to extend outwardly via a needle exit hole of the main body housing to an actuated position, and this step is implemented by, for example, a needle control component.

From the above discussion, it can be seen that in the present invention, the needle control component controls the access needle in two main aspects: control of the orientation of the access needle in an initial position, which enables at least partial communication between the suture carrying portion of the access needle and the suture slot to allow loading of the suture into the suture carrying portion via the suture slot; and control of the actuation depth and the orientation of the access needle in a moving process from the initial position to the actuated position, which enables the access needle to accurately reach the actuated position at a predetermined depth and radial position.

A tissue closure device and an access needle of the present invention form an integrated unit which can be delivered to an operator conveniently. In the present invention, the unique arrangement of an access needle and a suture slot of a main body housing enables an operator to conveniently load a suture into the access needle and release the suture from the main body housing, and also facilitates exit of the access needle carrying the suture from the tissue closure device. The entire operating process is fast and the control over the access needle is accurate, thus achieving a better operation effect. In a suture loading process, the tip of the access needle is concealed in the main body housing, so that an operator can be protected from a puncture wound, and the tip of the access needle is less likely to be contaminated.

From the above, those skilled in the art will readily recognize that alternative structures of the structures disclosed in the present invention may be used as feasible alternative embodiments, and the embodiments disclosed in the present invention may be combined to form new embodiments, which also fall within the scope of the appended claims.

1. A tissue closure device, comprising:
a main body housing having a longitudinal axis, the main body housing internally defining a chamber extending along the longitudinal axis, and a suture slot and a needle exit hole being provided on a sidewall of the main body housing; and
an access needle, which is capable of being preloaded in the chamber, is provided with a suture carrying portion close to a tip thereof, is capable of being held in an initial position, and is capable of being actuated distally from the initial position to an actuated position,
wherein when the access needle is in the initial position, the access needle is accommodated in the chamber, and the suture carrying portion and the suture slot are at least partially communicated to enable a suture to enter the suture carrying portion via the suture slot; and when the access needle is in the actuated position, the access needle penetrates through the needle exit hole, and the tip of the access needle is located outside the chamber.

2. The tissue closure device according to claim 1, wherein the suture slot comprises a suture loading slot and a suture releasing slot, the suture loading slot being configured to allow the suture to be loaded from the outside of the tissue closure device into the suture carrying portion of the access needle via the suture loading slot, and the suture releasing slot being communicated between the suture loading slot and the needle exit hole and configured to allow the suture to disengage from the tissue closure device via the suture releasing slot, wherein the suture loading slot and the suture carrying portion are at least partially aligned when the access needle is in the initial position.

3. The tissue closure device according to claim 1, wherein the suture slot is an integrated slot structure serving as both a suture loading slot and a suture releasing slot, the suture slot being configured to allow the suture to be loaded from the outside of the tissue closure device into the suture carrying portion of the access needle via the suture slot and to allow the suture to disengage from the tissue closure device via the suture slot.

4. The tissue closure device according to claim 2, wherein the suture loading slot has a first slot portion and a second slot portion, the first slot portion extending at least partially towards the axis from the outside to the second slot portion, and the second slot portion extending at least partially axially from the first slot portion along a direction away from the needle exit hole, wherein the second slot portion and the suture carrying portion are at least partially aligned.

5. The tissue closure device according to claim 4, wherein the first slot portion constitutes an elongated opening along a direction at least partially perpendicular to the longitudinal axis, two tips of the elongated opening respectively extending along a direction away from the needle exit hole and towards the axis to jointly define the second slot portion.

6. The tissue closure device according to claim 4, wherein the extending directions of the suture releasing slot and the second slot portion are the same.

7. The tissue closure device according to claim 1, wherein a cutting surface that constitutes a guide structure is provided at an inlet of the slot on the sidewall of the main body housing.

8. (canceled)

9. The tissue closure device according to claim 1, wherein two perforated structures that are symmetrical about the longitudinal axis are provided on the sidewall of the main body housing, each of the perforated structures comprising at least one said suture slot and at least one said needle exit hole.

10. The tissue closure device according to claim 1, wherein the main body housing comprises a non-rigid suture retaining portion configured to be capable of contacting with the suture and holding the suture in place by friction.

11. The tissue closure device according to claim 10, wherein the main body housing comprises a rigid sidewall having a window formed thereon, the suture retaining portion being mounted at the window and constituting a part of the sidewall of the main body housing, wherein at least a part of the suture slot is at least formed on the suture retaining portion.

12. The tissue closure device according to claim 11, wherein the suture slot comprises a suture loading slot, and a suture releasing slot communicated between the suture loading slot and the needle exit hole, wherein at least one of the following conditions is present:
at least a part of the suture loading slot is formed on the suture retaining component; or
at least a part of the suture releasing slot is formed on the suture retaining portion.

13. The tissue closure device according to claim 11, wherein a protrusion portion protruding along the sidewall of the main body housing is disposed on the suture retaining portion, a corresponding recess portion is provided at the window of the rigid sidewall, and the protrusion portion and the recess portion cooperate to secure the suture retaining portion and the rigid sidewall.

14. The tissue closure device according to claim 11, wherein a protrusion portion protruding along the sidewall of the main body housing is provided at the window of the rigid sidewall, a corresponding recess portion is provided at the edge of the suture retaining portion, and the protrusion portion and the recess portion cooperate to secure the suture retaining portion and the rigid sidewall.

15. The tissue closure device according to claim 11, wherein the portion serving as the suture loading slot of the suture slot is at least partially formed on the suture retaining portion, and has a width less than or equal to the width of the suture.

16. The tissue closure device according to claim 10, wherein the main body housing comprises a rigid sidewall, the suture retaining portion is mounted on the rigid sidewall and is independent of the suture slot, and the suture retaining portion is configured to allow the suture to enter between the suture retaining portion and the rigid sidewall so as to clamp the suture between the suture retaining portion and the rigid sidewall.

17.-18. (canceled)

19. The tissue closure device according to claim 16, wherein a recessed portion is provided on the rigid sidewall, the suture retaining portion is inserted into the recessed portion with the shape thereof fit to the same, and the outer surface of the suture retaining portion is flush with the outer surface of the rigid sidewall.

20. The tissue closure device according to claim 19, wherein the suture retaining portion comprises a central portion and end portions located at both ends of the central portion, the inner surface of the central portion protruding further inwardly toward the longitudinal axis relative to the inner surfaces of the end portions.

21.-23. (canceled)

24. The tissue closure device according to claim 1, wherein a recessed and externally open suture accommodating portion that extends parallel to the longitudinal axis is provided on the sidewall of the main body housing, the suture accommodating portion being connected between the suture slot and a suture winding portion for winding the suture.

25.-26. (canceled)

27. The tissue closure device according to claim 1, wherein the tissue closure device comprises a stabilizer mounted at one end of the main body housing and actuatable between a folded position and an unfolded position, and when the access needle is in the actuated position, the access needle penetrates through the stabilizer in the unfolded position.

28. A method of using the tissue closure device according to claim 1, comprising:
a suture loading step comprising: loading a suture into a suture carrying portion of an access needle via a suture loading slot of a main body housing; and
a needle exiting step comprising: causing the access needle loaded with the suture to extend outwardly from a needle exit hole of the main body housing to an actuated position.
